(19) United States
(12) Patent Application Publication
(10) Pub. No.: US 2024/0331817 A1
(43) Pub. Date: Oct. 3, 2024

(54) SYSTEMS AND METHODS FOR MONITORING PATIENTS AND ENVIRONMENTS

(71) Applicant: Welch Allyn, Inc., Skaneateles Falls, NY (US)

(72) Inventors: John A. Lane, Venice, FL (US); David E. Quinn, Auburn, NY (US); WonKyung McSweeney, Manlius, NY (US); Danielle Endres, Syracuse, NY (US); Gene J. Wolfe, Pittsford, NY (US)

(73) Assignee: Welch Allyn, Inc., Skaneateles Falls, NY (US)

(21) Appl. No.: 18/616,007

(22) Filed: Mar. 25, 2024

Related U.S. Application Data
(60) Provisional application No. 63/455,219, filed on Mar. 28, 2023.

(51) Int. Cl. G16H 10/60; G06V 10/774; G06V 10/776; G06V 20/40; G06V 20/52; G06V 20/70; G06V 40/10; H04N 7/18

(52) U.S. Cl. CPC G16H 10/60; G06V 10/774; G06V 10/776; G06V 20/44; G06V 20/52; G06V 20/70; G06V 40/10; H04N 7/181

(57) ABSTRACT

A system and method for identifying events occurring in a patient room during an interaction with a caregiver, generating, using a machine learning model, an event description based on video data from the patient room, and recording the description with annotations to the electronic medical record of the patient is described. The system and method use one or more cameras and/or sensors within a room of a patient to detect events and interactions and uses machine learning techniques to identify the events and extract notes for inclusion in the electronic medical record for tracking and providing care to a patient in a care facility

### CROSS-REFERENCE TO RELATED APPLICATIONS

This application claims priority to U.S. Provisional Patent Application No. 63/455,219, filed Mar. 28, 2023, titled “SYSTEMS AND METHODS FOR MONITORING PATIENTS AND ENVIRONMENTS,” the entire disclosure of which is incorporated herein by reference.

### FIELD OF THE INVENTION

The present disclosure relates to systems and methods for monitoring patients, environments, equipment, caregivers, and other items in a caregiver setting.

### BACKGROUND OF THE INVENTION

In patient care environments, such as hospitals, nursing homes, rehabilitation facilities, skilled nursing facilities, post-surgical recovery centers, and the like, caregivers employ a variety of medical devices (for example, physiological sensors) that interact with patient monitoring devices which display a significant amount of patient health information. Such information is typically displayed on handheld monitoring devices or stationary monitoring devices with limited visual “real estate.” Often if not always, multiple patients are being monitored at once. Further, such health information is constantly fluctuating for multiple patients in a simultaneous manner, increasing the difficulty for a caregiver to locate, evaluate, and respond to a particular piece of health information for a particular patient. Additionally, charting of patient data and information can be difficult given staffing limitations that require a small number of caregivers to enter patient data into electronic medical records (EMRs). Because caregivers are under significant time pressure and only have a small amount of time to monitor, respond to, and/or treat individual patients under their care, it is difficult for caregivers to quickly obtain information regarding a patient's status at any given time, let alone evaluate such information and determine if the patient's care plan or EMR needs to be updated. Even the slightest speed advantage for caregivers in such situation can greatly reduce burdens to providing superior care and accurately reflect all of the care and procedures provided to a patient.

The example embodiments of the present disclosure are directed toward overcoming the deficiencies described above.

### SUMMARY

In some examples, the systems and techniques described herein provide a system including a camera positioned in a room of a patient care facility, one or more processors, and one or more non-transitory, computer-readable media having instructions stored thereon that, when executed by the one or more processors, cause the one or more processors to performs one or more acts. The acts may include receiving video data from the camera of the room including a representation of a patient and determining, using a machine learning model trained using training data including annotated video data of patient care facilities with annotations of event data, an event occurring within the room. The acts may also include generating, using the machine learning model, an annotation associated with the event, and updating, in response to the event and based on the annotation, an electronic medical record of the patient.

In some examples, the camera may include a two-way camera system that enables communication between the room of the patient care facility and a caregiver station. The system may, in some examples, include one or more medical devices positioned within the room and configured to collect patient data. The annotation may be generated based on the patient data from the one or more medical devices. Receiving the patient data may include determining, based on the video data, that a display associated with the one or more medical devices is presenting a representation of the patient data and determining the patient data from the video data and the representation of the patient data. Determining the event may include determining a care procedure from a care plan for the patient, and the acts may include determining, based on the electronic medical record, a prescribed procedure for the patient. The system may also determine a compliance score based on the event and the prescribed procedure and updating the electronic medical record based on the compliance score. The acts may also include receiving second video data from the second camera of the room including a second patient, determining, using the machine learning model, a second event occurring within the second room, generating, using the machine learning model, a second annotation associated with the second event, and updating, in response to the second event and based on the second annotation, a second electronic medical record of the second patient. The acts may also include displaying, at the display of the caregiver station, video data representing the event, the annotation, video data of the second video data representing the second event, and the second annotation. The acts may also include receiving an input via the input device, and updating the electronic medical record or the second electronic medical record in response to the input. The acts may also include determining, using the machine learning model, a confidence score associated with the event; and requesting a user input in response to the confidence score being below a threshold.

In some examples, the systems and techniques described herein provide a method for monitoring and recording patient care activities in an automated manner in a patient care facility. The method includes receiving, at a computing device associated with a care facility, video data from a camera positioned within a patient room of the care facility. The method also includes determining, using a machine learning model trained using training data including annotated video data of patient care with annotations of event data, an event occurring within the room. The method further includes generating, using the machine learning model, an annotation associated with the event. The method also includes updating, in response to the event and based on the annotation, an electronic medical record of the patient. Other embodiments of this aspect include corresponding computer systems, apparatus, and computer programs recorded on one or more computer storage devices, each configured to perform the actions of the methods.

In some examples, the method may also include displaying at a caregiver station of the care facility, a representation of the video data associated with the event, the electronic medical record, and the annotation. The method may also include determining a confidence score associated with the event and requesting a user input in response to the confidence score being below a threshold, the user input requested to confirm identification of the event. In some examples, the event may include identification of a person in the room, and the annotation may include a reference to the identification of the person. The identification of the person may be determined by accessing the video data, determining a unique identifier associated with the person visible in the video data, and determining the identification of the person based on the unique identifier. The annotation may include data representing the interaction. Implementations of the described techniques may include hardware, a method or process, or computer software on a computer-accessible medium.

### BRIEF DESCRIPTION OF THE DRAWINGS

FIG. 1 illustrates an example system for patient monitoring within a patient care environment, according to at least one example.

FIG. 2 illustrates an example system for identifying individuals in a patient care environment, according to at least one example.

FIG. 3 illustrates an example system architecture for patient monitoring and charting in a patient care environment, according to at least one example.

FIG. 4 illustrates an example system architecture for monitoring multiple patient areas in a patient care environment, according to at least one example.

FIG. 5 illustrates an example system architecture for monitoring and centralizing patient care data, according to at least one example.

FIG. 6 illustrates an example process for charting patient information to an electronic medical record using the systems described herein, according to at least one example.

FIG. 7 illustrates an example user interface for a patient care monitoring hub, according to at least one example.

FIG. 8 illustrates an example process for automatic charting of patient information to an electronic medical record, according to at least one example.

FIG. 9 illustrates a block diagram of a computing system, according to at least one example.

### DETAILED DESCRIPTION

Many virtual nursing systems, or other patient care systems, rely on a camera, audio, screen on both the patient side and caregiver station. While the patient views the interaction as a 1-to-1 interaction, the caregiver (e.g., virtual nurse station) is set up as 1-to-many. Virtual caregivers may include nurses that have practiced in a particular setting and have experience with charting, admission, and education in the care facility. However, in a 1-to-many setting on the virtual caregiver side, manually taking down notes about the patients and patient care for charting is time consuming and labor intensive, requiring staff to manually enter annotations rather than spend time focused on caregiver activities that directly interact with patients. Moreover, there may be many notes, annotations, insights, clinical contexts, and other such information that may be lost in charting due to current limitations of manual annotations and time constraints to exhaustively chart information related to a patient.

The systems and methods described herein provide for automated charting of events related to a patient in a caregiver facility. The charting of events and information described herein may include charting information to the EMR of the patient. Charting the events and/or data may include automatically updating the EMR to include the new data, overwriting and/or replacing existing or conflicting data. Charting may include generating data that can be added to the EMR or that may be included with other information manually added to the EMR of the patient. In some examples, the system may automatically chart (e.g., update and/or save new data) the information to the patient EMR. In some examples, the system may generate charting data that can be associated with the EMR without explicitly being added to the EMR, for example by linking the data to the EMR for access by caregivers or other systems through the EMR.

The systems and methods herein provide for virtual caregiver systems and methods ensuring maximum usage of technology while ensuring patient safety and providing an improved charting of patient information to aid in providing superior care to the patients. In particular, the systems and methods herein provide for resolving a shortfall in caregiver facilities by enabling detailed tracking and logging of patient data without requiring extensive manual labor that is time intensive and costly, especially with caregivers in short supply.

As described herein, the caregiver station enables viewing and monitoring of various patient rooms. Accordingly, the system may receive video data from multiple rooms, determining events associated with each patient, and recording annotation information to the EMR of the corresponding patient.

In an example, this description provides for automated charting of clinical data for patients that may be autonomously generated as events are detected and/or observed by sensors of the caregiver facility. In the example, the patient room of a caregiver facility may be equipped with a camera. The camera may be used to capture image and/or video data that may be analyzed to determine events occurring within the room. In some examples, additional sensors and devices may output data that may be used, in conjunction with the data from the camera, to determine events. The events may occur an action taken by a caregiver, the patient, a visitor, or other individual. The events may occur providing treatment, adherence with a prescribed treatment, or other such information. The events identified may include identification of an individual (e.g., a doctor or nurse) as well as an identification of the action (providing a treatment, giving instruction, etc.).

In some examples, the events, or a set of events to choose from may be stored in an event catalog. The event catalog may include a set of events that can be defined using the video data, device data, patient data, or other sensor data. The event catalog may not be exhaustive, but may grow over time as additional events and event types are added.

Identification and tracking of patients and individuals within the patient room of the care facility enables the system to track within video and other sensor data and, using machine learning models, identify events (involving individuals or involving only the patient). Additionally, due to the ability to enroll and/or identify individuals, charting events involving clinicians may be recorded, with charting events including any events that may be recorded on a chart of a patient. In some examples, the system may automatically identify individuals based on visual characteristics, such as using facial recognition and/or identification of a visual marker (such as an ID badge) of the individual. In this manner a doctor, nurse, or other caregiver may be identified to associate with a particular event. The identification may enable the system to identify authentication and authorization for events, care procedures, charting events, and other actions. Additionally, the identification systems may be used for identifying objects, such as medical devices, treatment devices, medications, and other objects within the room of the patient for recording data, such as when treatments are administered, settings for treatments, and other such data.

The events, and associated data, may be automatically charted to an electronic medical record (EMR) of the patient. In some examples particular systems may be configured to perform tasks described herein. For example, a charting agent may perform automated charting based on the identified events and data from the sensors of the patient room. In some examples, additional agents such as an alerting agent may trigger alerts if a detected event requires alerting clinician or care providers. In some examples, a recording agent may record when events are detected, along with associated annotations of the events.

In addition to identification of individuals, identification of events, tracking of individuals, tracking of care provided to a patient, and charting of information to the EMR, the system provides for monitoring from a caregiver station to quickly view current and relevant information related to each patient under the care of the caregiver station. The caregiver station may include a display that provides a view of the EMR data for the several patients as well as associated data, such as video data, annotations, recent events identified, and other such information that may be instructive to the caregiver. The caregiver may, in this manner, be able to monitor a number of patients efficiently and safely while also ensuring that the records and charts are kept up to date and accurate in a manner not previously possible.

In some examples, the systems and techniques described herein use one or more sensors and/or devices within a patient room of a care facility as well as one or more computing devices to enable the benefits described herein. For example, the camera and/or other sensors may gather sensor data that is conveyed to the computing device. The computing device may determine, using one or more machine learning models, an event occurring within the room as well as associated annotations. The machine learning model may be trained using training data including annotated video data of patient care with annotations of event data. In some examples a first machine learning model may be used to identify events within the room, a second machine learning model may be used for object detection of relevant objects associated with the events (e.g., equipment used or interacted with), a third machine learning model may identify individuals based on enrollment data, and a fourth machine learning model may generate an annotation describing the event and/or other data for entry into the EMR of the patient.

The camera may include a two-way camera system that enables communication between the room of the patient care facility and a caregiver station thereby enabling the caregiver station to interact with and view various patient room data from a single hub. The caregiver station may be used for providing alerts when action is needed, to review charting of events automated by the system, and to approve or review events charted to the EMR of the patient.

In some examples, the patient room may include one or more additional devices, such as medical devices, computing devices, and other objects. The devices may, in some examples be equipped with communication devices capable of communicating data to the computing system associated with the caregiver station. In this manner, medical devices may provide accurate and complete data, for example including pulse, blood oxygenation, therapeutic drug delivery rate and amount, blood pressure, or any other suitable information. After an event is identified by the system, such as based on the image data from the camera, the data from the medical devices may be used to generate an annotation of the event describing the state of the patient in detail. In some examples, the data from the medical devices may be added to the EMR without being associated with an event from the video data.

In some examples, the devices or objects within the patient room may not be equipped with communication devices to deliver data to the computing system. In such examples, the system may leverage the one or more cameras to determine data associated with the device. For example, a medical device having a display may be visible to the camera and the computing device may be configured to determine the data from the medical device based on the information displayed at the display of the device. In a particular example, a type of patient monitor may include blood pressure data, but the monitor may not be equipped to communicate the data directly to the computing system. Instead, in a typical example, a caregiver would have to manually enter the data into the EMR. Instead, the system may automatically determine, from the display of the device, the blood pressure data and chart to the EMR. In this manner, the problem of accurately gathering and recording patient data in an efficient and safe manner is provided by the solution described herein.

In some examples, the system may determine a prescribed care plan for a patient, such as a procedure, dosage, or regimen for a patient to follow. The care plan may be determined from the EMR for the patient. For instance, a doctor may, while discussing with the patient, describe the care plan. The system may use a microphone to pick up audio data of the care plan and record the care plan to the EMR as prescribed by the doctor. Subsequently, the system may identify patient compliance with the care plan based on sensor data. If the patient does not adhere to the care plan the EMR may reflect the discrepancy such that the caregiver is aware of the progress with respect to the care plan prescribed by the caregiver. In some examples, the system may also determine a compliance score with the care plan indicative of a level of compliance with following the instructed care plan. The compliance score may be presented at the caregiver station for viewing by the caregiver, for example to identify patients that may require additional monitoring or care.

In some examples, the event data, annotations, identification of individuals, or other data produced by the system may have an associated confidence score. The confidence score may be reflective of the confidence of the system in the produced data. In some instances, the system may present, for manual confirmation, data with a corresponding confidence threshold that falls below a specified level. For example, if the system has less than an 80% confidence in a particular event (such as a doctor administering care), the system may present an alert to the caregiver station indicating the event data as well as the associated video data. The caregiver may briefly review and provide an input to confirm or reject the event data. This additional input and confirmation may be used by the system to continue learning of events, objects, identities, and other data using machine learning models.

In some examples, particular events may require caregiver authorization or verification by a second party. For example, performing a blood transfusion, medication administration or disposal for certain medications, or other such events may require verification due to the nature of the events or instructions by the care facility, which may customize what events require confirmation.

In some examples, the determinations and computations described herein may be performed locally, at a computing device local to the patient room, and conveyed to a caregiver station or may be conveyed to a single computing system where all patient data is processed. The computing systems may remain on-premises for protecting private healthcare information of individuals. In some examples, the patient data may be anonymized and/or encrypted when processed by the computing device, with a code, key, or tag, that may be used to decode the patient identifier information after processing. In this manner, the patient privacy and security of health data may be preserved.

In some examples, the system may be used for hazard identification and determination. For instance, object detection using machine learning models may be used to identify potential hazards within the room, or to identify obstacles that may impede a path of a patient. In addition to environmental hazards, the system may identify hazards for the patient. For example, pose detection of the patient using a machine learning model may enable tracking of the position and pose for the patient within the care facility. Additionally, detection of hazards, such as slips, falls, limping, or other such injuries may be detected using the tracking and pose detection capabilities of the system. Alerts may also relate to alerts of problems or potential problems. For instance, the system may identify and intravenous drug delivery system including an IV line that goes to the arm of a patient. The system may identify, based on the video data, when the IV line becomes detached from the arm or hand of the patient using object detection techniques. Accordingly, the system may note the deviation and alert eh caregiver to remedy the situation.

In an additional illustrative example, a patient room may be equipped with a camera system, among other sensors. The system, via the camera system, may receive video and/or audio data representing the caregiver and create an annotation associated with the video data. For example, a doctor may perform a test on the patient and the test and results thereof may be captured by the video camera system and/or other system. The system may identify the event based on the data from the camera and generate annotation data, using one or more machine learning models describing the event. The video data may be streamed to the caregiver station in real-time, and also available after real-time playback for review and confirmation in the event that the caregiver was not available at the station in real-time. The caregiver may review the generated annotation and approve, if required, or adjust as needed to accurately reflect the event. Such data may be used to refine the machine learning model to produce more accurate annotations of events. In such examples, the machine learning model may be configured for continuous learning and refinement of the models to produce annotations for EMRs based on the refined data. In the case of audio data, the voice of the caregiver may be used to authenticate the source, using a voice identity. In the case of video data, the appearance and/or visible credentials/identifier associated with the caregiver may be used for authorization.

For instance, the caregiver may us a “wake word”, such as “chart BP145/80.” The system may repeat back the data as understood according to the event as “chart BP systolic 145/80.” The caregiver may then confirm or adjust the data before it is added to the EMR of the patient. In some examples, the caregiver may dictate information to be charted, and one or more microphones or audio sources within the room may capture the audio data to be transcribed and added to the EMR. In this manner, the caregiver may dictate notes to add to the EMR. In some examples, the system may transcribe, using an algorithm (e.g., a natural language processing algorithm), the audio data to generate and/or determine transcription data. The transcription data may be used to generate one or more annotations to add to the EMR.

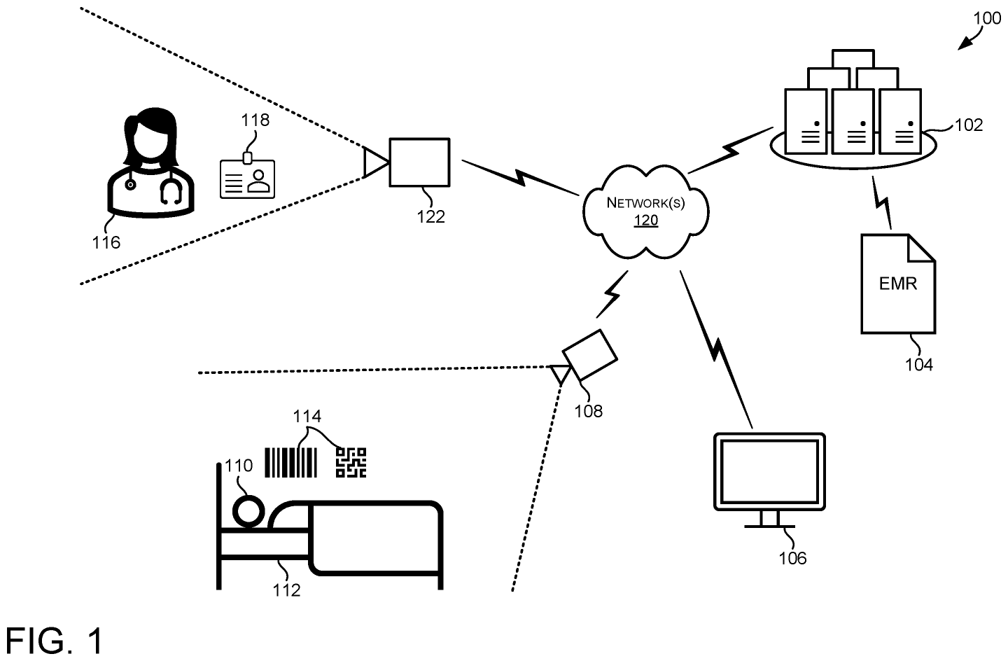

Turning to the figures, FIG. 1 illustrates a system 100 for patient monitoring within a patient care environment, according to at least one example. In the system 100, a computing device 102 may perform various actions as described herein. The computing device 102 may be located on-site at a caregiver facility. The computing device 102 of FIG. 1 is shown as storing and/or having access to an EMR 104 for a patient 110. the EMR 104 stores patient data and care information for the patient 110. The computing device 102 also communicates over network(s) 120 with one or more additional devices. The additional devices include a caregiver station 106, a camera 108, and one or more additional cameras 122. In some embodiments, the network(s) 120 may be any type of network known in the art, such as the Internet, a wireless Local Area Network (LAN), or a wide-area network (WAN). Moreover, the computing device 102 and the other components of FIG. 1 may communicatively couple to the network(s) 120 in any manner, such as by a wired or wireless connection. The network(s) 120 may also facilitate communication between the computing device 102 and a database storing the EMR 104.

In the example of FIG. 1, the cameras 108 and 122 are used for detecting identities of individuals for tracking within the caregiver facility. As described herein, events that may be observed and recorded on the EMR may be associated with a caregiver 116 and will be associated with a patient 110. The camera 108, for example may be positioned within a room of a patient 110 and capture a view of the patient 110, a bed 112, and other objects within the room. The camera 108 may be supplemented by additional cameras or other sensors to detect data of the patient 110 and the room.

The patient 110 may be associated with a unique identifier that is displayed, included in, indicated by, and/or otherwise provided by a code 114. The code 114 may include any suitable visible symbol for encoding information, such as a barcode, quick-read (QR) code, alphanumeric string, or other visual identifier. The code 114 may be visible within the room, for example attached to the foot of the bed 112 and/or on a wristband or badge worn by the patient 110. Accordingly, the patient 110 may be identified using data from the camera 108 that includes a representation of the patient as well as the code 114. Therefore, as events are recorded or detected by the camera 108 and/or other sensors within the room of the patient, they may be correctly associated with the EMR 104 of the patient 110.

Identification of the patient 110 within the room may enable the computing device 102 to track the patient 110 as they move and are within range of the camera 108 and other sensors. Additionally, other individuals may be identified by the camera 108. Accordingly, events may be properly associated with the individuals involved, for example to include instructions or charting information provided by a caregiver 116 relative to a patient 110, the camera 108 may capture video data such that the computing device 102 can identify each of the patient 110 and caregiver 116 for charting to the EMR.

The identification of patient 110 and caregiver 116 may use one or more machine learning techniques for object and/or person recognition. The computing device 102 may house one or more machine learning models to perform such tasks. To aid with the identification, patients and caregivers may have the visual identifiers that may be visible to the camera and/or be enrolled with a facial recognition system. For example, camera 122 may be used to enroll patients 110 and/or caregivers 116. The camera 122 may capture image data of the caregiver 116 and associated credentials and/or identifiers included in the ID 118. The ID 118 may be associated with a caregiver profile stored in association with the computing device 102 such that when the caregiver 116 is enrolled, they may be readily identified by the computing device 102 for various purposes, with or without the ID 118. However, the initial enrollment may, in some examples, rely on the ID 118 for identification of the caregiver 116.

The identification and tracking of patients and individuals within the patient room of the care facility enables the computing device 102 to track individuals within video and other sensor data and, using machine learning models, identify events (involving individuals or involving only the patient). Additionally, due to the ability to enroll and/or identify individuals, the computing device may chart events involving caregiver 116, with charted events including any events that may be recorded on a chart of a patient 110. In some examples, the computing device 102 may automatically identify individuals based on visual characteristics, such as using facial recognition and/or identification of a visual marker (such as ID 118 or code 114) of the individual. In this manner a doctor, nurse, or other caregiver may be identified to associate with a particular event. The identification may enable the system to identify authentication and authorization for events, care procedures, charting events, and other actions. Additionally, the identification systems may be used for identifying objects, such as medical devices, treatment devices, medications, and other objects within the room of the patient for recording data, such as when treatments are administered, settings for treatments, and other such data.

The system 100 provides for automated charting of events related to the patient 110 in a caregiver facility to record the events to the EMR 104. The system 100 provides for a virtual caregiver system ensuring maximum usage of technology while ensuring patient safety and providing an improved charting of patient information to aid in providing superior care to the patients. In particular, the system 100 resolves a shortfall in caregiver facilities by enabling detailed tracking and logging of patient data without requiring extensive manual labor that is time intensive and costly, especially with caregivers in short supply.

In an example, this description provides for automated charting of clinical data as events for the patient 110 that may be autonomously generated as events are detected and/or observed by camera 108 or other sensors of the caregiver facility. The camera 108 may be used to capture image and/or video data that may be analyzed to determine events occurring within the room. In some examples, additional sensors and devices may output data that may be used, in conjunction with the data from the camera 108, to determine events. The events may represent an action taken by a caregiver 116, the patient 110, a visitor, or other individual. The events may occur providing treatment, adherence with a prescribed treatment, or other such information. The events identified may include identification of an individual (e.g., a doctor or nurse) as well as an identification of the action (providing a treatment, giving instruction, etc.).

In some examples, the events, or a set of events to choose from may be stored in an event catalog. The event catalog may include a set of events that can be defined using the video data, device data, patient data, or other sensor data. The event catalog may not be exhaustive, but may grow over time as additional events and event types are added.

In an example, a doctor may perform a test on the patient and the test and results thereof may be captured by the video camera system and/or other system. The system may identify the event based on the data from the camera 108 and generate annotation data, using one or more machine learning models describing the event. The video data may be streamed to the caregiver station 106 in real-time, and also available after real-time playback for review and confirmation in the event that the caregiver was not available at the station in real-time. The caregiver 116 may review the generated annotation and approve, if required, or adjust as needed to accurately reflect the event. Such data may be used to refine the machine learning model to produce more accurate annotations of events.

The caregiver 116 may, for example, while performing a blood pressure measurement on the patient 110 indicate, audibly, that the “blood pressure is 145/80.” The computing device 102 may use the video and/or audio data to identify the event of testing the blood pressure as well as the result as provided by the caregiver 116. The caregiver 116 may confirm the blood pressure value while in the room or back at the caregiver station 106. In the EMR 104, the computing device will store data related to the timestamp, the blood pressure indicated, the identity of the caregiver 116, the event (e.g., blood pressure test), and other associated data such as video and audio data may be stored or linked to from the EMR.

The events, and associated data, may be automatically charted by the computing device 102 to the EMR 104. In some examples particular systems or modules of the computing device 102 may be configured to perform tasks described herein. For example, a charting agent may perform automated charting based on the identified events and data from the sensors of the patient room. In some examples, additional agents such as an alerting agent may trigger alerts if a detected event requires alerting clinician or care providers. In some examples, a recording agent may record when events are detected, along with associated annotations of the events.

In addition to identification of individuals, identification of events, tracking of individuals, tracking of care provided to a patient 110, and charting of information to the EMR 104, the system provides for monitoring from a caregiver station 106 to quickly view current and relevant information related to each patient under the care of the caregiver station. The caregiver station 106 may include a display that provides a view of the EMR 104 data for the several patients as well as associated data, such as video data, annotations, recent events identified, and other such information that may be instructive to the caregiver 116. The caregiver 116 may, in this manner, be able to monitor a number of patients efficiently and safely while also ensuring that the records and charts are kept up to date and accurate in a manner not previously possible.

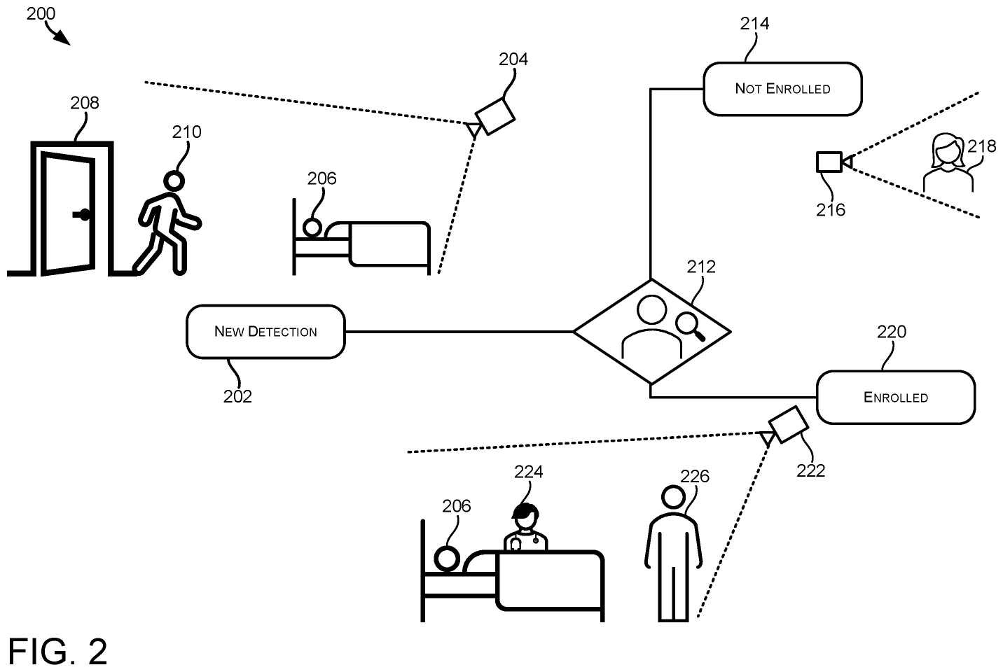

FIG. 2 illustrates a system 200 for identifying individuals in a patient care environment, according to at least one example. As described with respect to FIG. 1, the computing device 102 may chart data for patients automatically and record and track individuals to include with the charting data. In some examples, individuals may enter the room of the patient that are not enrolled with the computing device 102. For example, though the patient 110 and caregiver are enrolled and equipped with visible markers and/or enrolled in person recognition by the computing device 102, a person 210 may enter the patient room at door 208 and the person 210 may not be enrolled in the system.

The computing device 102 may detect the person 210 with camera 204 at a new detection 202. The computing device 102 may then search enrolled individuals based on the new detection 202. In the event that the person 210 is enrolled (e.g., a caregiver, visitor who previously enrolled, or patient), then the computing device 102 may identify them as enrolled 220 and proceed as described herein. In some instances, the person 210 may not be found by the computing device 102 and therefore is not enrolled 214. Upon being detected as not enrolled 214, the computing device 102 may perform an automatic enrollment for the person 210. The automatic enrollment may include capturing image data using camera 216 (which may be the same as camera 204 or a different camera) of person 218 for use in identifying and tracking through the caregiver facility. The computing device 102 may assign the person 218 a unique identifier such that they may be uniquely identified within the data captured and processed by the computing device 102, even without a known identity. In this manner, if or when the person 218 is eventually identified, the computing device 102 may update the unique identifier with the identifying information and update all references to the person 218 accordingly.

For example, a camera 4122 may capture an example patient room for a patient 206 while two other individuals are present. One of the individuals may be a caregiver 224 who is enrolled and therefore readily identified by the computing device 102. The other individual 226 may be a visitor or other unenrolled person. Accordingly, the system may only chart, to the EMR, information from the caregiver 224 and patient 206 while not charting information from the other individual 226. Accordingly, as the caregiver 224 performs tests or interacts with the patient, the information may be determined by a machine learning model of the computing device 102 and charted to the EMR 104. Information associated with the other individual 226 may be recorded by the computing device 102 in some examples, but may not be charted to the EMR 104. Instead, such data may be stored separately. Therefore, if, at a later time the other individual 226 is identified as a caregiver, then the previous interactions may be charted to the EMR 104. In some examples, the EMR may include reference to the other individual 226 regardless of identification as a caregiver.

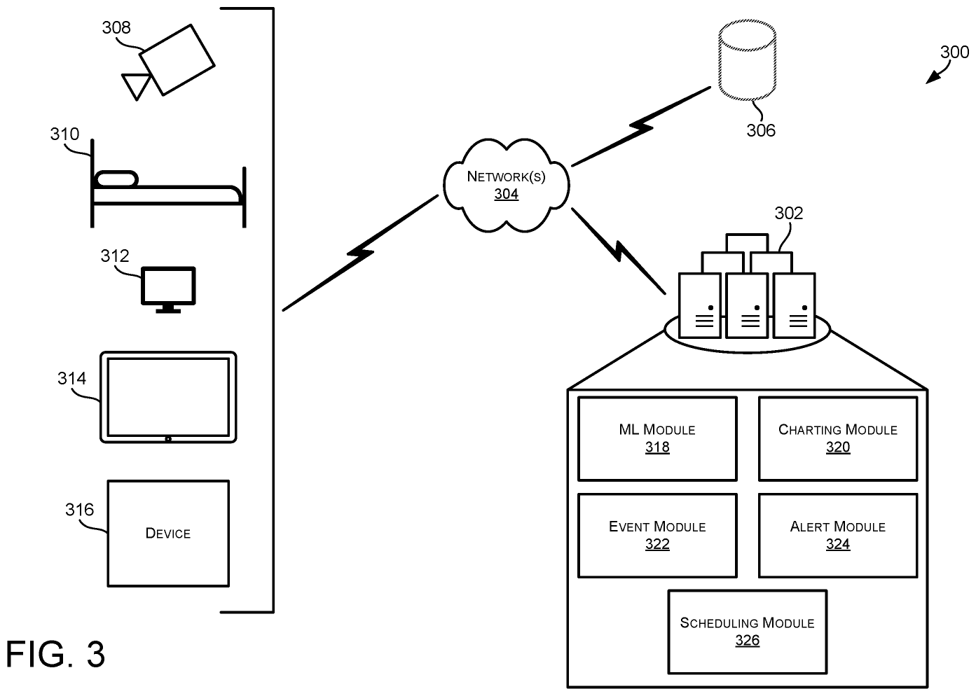

FIG. 3 illustrates a system architecture 300 for patient monitoring and charting in a patient care environment, according to at least one example. The system architecture 300 includes a computing device 302, which may be similar or identical to the computing device 102 as well as various devices that provide data to the computing device 302 communicating over network 304, which may be same or identical to network(s) 120 of FIG. 1. The system architecture 300 also includes a database 306 that may store information such as EMR data for various patients and data such as identification enrollment for individuals. The devices include a camera 308, patient bed 310, patient monitor 312, two-way communication device 314, and one or more medical devices. The patient monitor may include a room station as part of a nurse call system such as the NAVICARE® Nurse Call system available from Hill-Rom Company®, Inc. of Batesville, Ind. Additional details of suitable systems for patient monitors can be found in U.S. Pat. Nos. 7,746,218; 7,538,659; 7,319,386; 7,242,308; 6,897,780; 6,362,725; 6,147,592; 5,838,223; 5,699,038 and 5,561,412 and in U.S. Patent Application Publication Nos. 2009/0217080 A1; 2009/0214009 A1; 2009/0212956 A1; and 2009/0212925 A1, each of which is hereby incorporated by reference herein in its entirety for all that it teaches to the extent not inconsistent with the present disclosure which shall control as to any inconsistencies.

In some embodiments, the devices are able to communicate wirelessly with specific components for this purpose. Alternatively, or additionally, locating tags may be attached to the devices. The locating tags include transmitters to transmit wireless signals to receivers or transceivers installed at various fixed locations throughout a caregiver facility. In some embodiments, the tags have receivers or transceivers that receive wireless signals from the fixed transceivers. For example, to conserve battery power, the locating tags may transmit information, including tag identification (ID) data, only in response to having received a wireless signal from one of the fixed transceivers. The fixed receivers or transceivers communicate a location ID (or a fixed receiver/transceiver ID that correlates to a location of a caregiver facility) to a locating server that is remote from the various fixed transceivers. Based on the tag ID and location ID received by the locating server, the locations of the various tagged equipment of the devices, the tag wearing caregivers, and the tag wearing patients is determined by the locating server.

The locations ID may be used in conjunction with the data from the camera 308 to aid in object detection and tracking through the patient room, as well as ensuring that patient data and information from the devices is correctly assigned to the patient and their EMR.

The graphical displays of the patient monitor 312, two-way communication device 314, and the one or more devices 316 may include status boards, graphical room stations, and mobile devices of caregivers and/or patients. The illustrative mobile devices of FIG. 3 are of a particular type, but the devices may also include pagers, PDA's, tablet computers, and the like. Status boards are oftentimes located at master nurse stations in healthcare facilities but these can be located elsewhere if desired, such as in staff breakrooms, hallways, and so forth.

The database 306 may include an electronic medical records (EMR) or health information systems (HIS) server communicatively coupled to the computing device 302.

The computing device 302 includes modules for performing various tasks, as described herein. Though shown with a particular arrangement and structure, the components of the computing device 302 may be arranged in any number of configurations to perform the tasks described herein. As illustrated, the computing device includes a machine learning module 318, a charting module 320, an event module 322, an alert module 324, and a scheduling module 326. In at least one configuration, the computing device 302 includes one or more processors and a non-transitory computer-readable media with memory that stores instructions for the modules.

The machine learning module 318 may include one or more machine learning models that may perform one or more tasks as described herein, including identification of individuals, identification of events, generation of annotations, determination of confidence scores, and other such processes described herein.

Machine learning may take empirical data as input, such as data from the manually classified audio, and yield patterns or predictions which may be representative of content-auxiliary characteristics associated with the audio and video data. Machine learning systems may take advantage of data to capture characteristics of interest having an unknown underlying probability distribution. Machine learning may be used to identify possible relations between observed variables. Machine learning may also be used to recognize complex patterns and make machine decisions based on input data. In some examples, machine learning systems may generalize from the available data to produce a useful output, such as when the amount of available data is too large to be used efficiently or practically. As applied to the present technology, machine learning may be used to learn which performance characteristics are preserved during a localization process and validate localized content when the performance characteristics are preserved.

Machine learning may be performed using a wide variety of methods of combinations of methods, such as contrastive learning, supervised learning, unsupervised learning, temporal difference learning, reinforcement learning and so forth. Some non-limiting examples of supervised learning which may be used with the present technology include AODE (averaged one-dependence estimators), artificial neural network, back propagation, Bayesian statistics, naive bayes classifier, Bayesian network, Bayesian knowledge base, case-based reasoning, decision trees, inductive logic programming, Gaussian process regression, gene expression programming, group method of data handling (GMDH), learning automata, learning vector quantization, minimum message length (decision trees, decision graphs, etc.), lazy learning, instance-based learning, nearest neighbor algorithm, analogical modeling, probably approximately correct (PAC) learning, ripple down rules, a knowledge acquisition methodology, symbolic machine learning algorithms, subsymbolic machine learning algorithms, support vector machines, random forests, ensembles of classifiers, bootstrap aggregating (bagging), boosting (meta-algorithm), ordinal classification, regression analysis, information fuzzy networks (IFN), statistical classification, linear classifiers, fisher's linear discriminant, logistic regression, perceptron, support vector machines, quadratic classifiers, k-nearest neighbor, hidden Markov models and boosting. Some non-limiting examples of unsupervised learning which may be used with the present technology include artificial neural network, data clustering, expectation-maximization, self-organizing map, radial basis function network, vector quantization, generative topographic map, information bottleneck method, IBSEAD (distributed autonomous entity systems based interaction), association rule learning, apriori algorithm, eclat algorithm, FP-growth algorithm, hierarchical clustering, single-linkage clustering, conceptual clustering, partitional clustering, k-means algorithm, fuzzy clustering, and reinforcement learning. Some non-limiting example of temporal difference learning may include Q-learning and learning automata. Another example of machine learning includes data pre-processing. Specific details regarding any of the examples of supervised, unsupervised, temporal difference or other machine learning described in this paragraph that are generally known are also considered to be within the scope of this disclosure. Support vector machines (SVMs) and regression are a couple of specific examples of machine learning that may be used in the present technology.

In some examples, the machine learning module 318 may include access to or versions of multiple different machine learning models that may be implemented and/or trained according to the techniques described herein. For example, the machine learning model may be trained using annotated video data of patient care facilities with annotations of event data describing events visible within the video data. The machine learning model may then be capable of receiving video data and outputting identifications and/or annotations of events contained or represented within the video data. The machine learning model may be continually updated and/or refined as additional types of events are added to the training data, for example, when a new procedure or task is added to a nurses workflow, the training data may be updated with video data of the procedure with associated annotations. Any suitable machine learning algorithm may be implemented by the machine learning module 318. For example, machine learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet50, ResNet101, VGG, DenseNet, PointNet, and the like.

The charting module 320 may maintain or be associated with one or more sources of data including data from the EMR, data from the devices, data from the camera, data from the one or more machine learning modules, and other sources. The charting module 320 may include one or more databases or access to one or more databases storing the EMR data and include the capability to write the EMR data of the patients.

The event module 322 may use one or more of the machine learning models from the machine learning module 318 to perform event determination tasks. For example, the machine learning models may include models for object recognition, audio speech recognition, person identification and the like for generating event data of events in the patient room based on data from the devices.

The alert module 324 may be configured to output or generate alerts, for example related to when a patient has an event that may be characterized as high risk, or is out of compliance with prescribed caregiver orders. For instance, as described herein, the computing device 302 may determine whether patient actions comply with orders from a caregiver and determine a compliance score. An alert may be generated if the compliance score drops below a threshold amount. In some examples, the alerts may be generated at the caregiver station and/or a device associated with a caregiver.

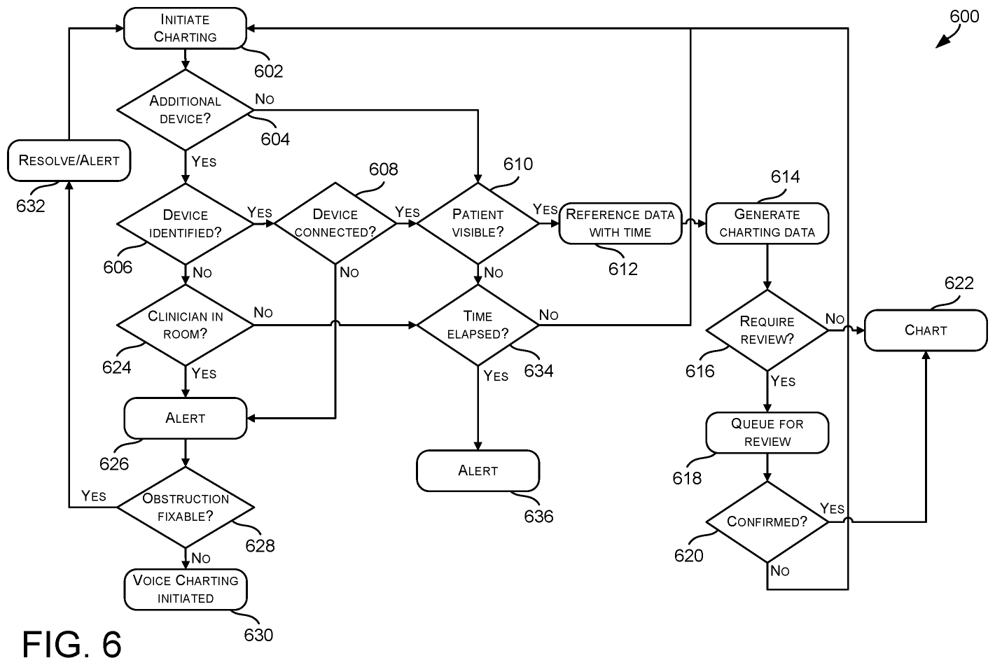

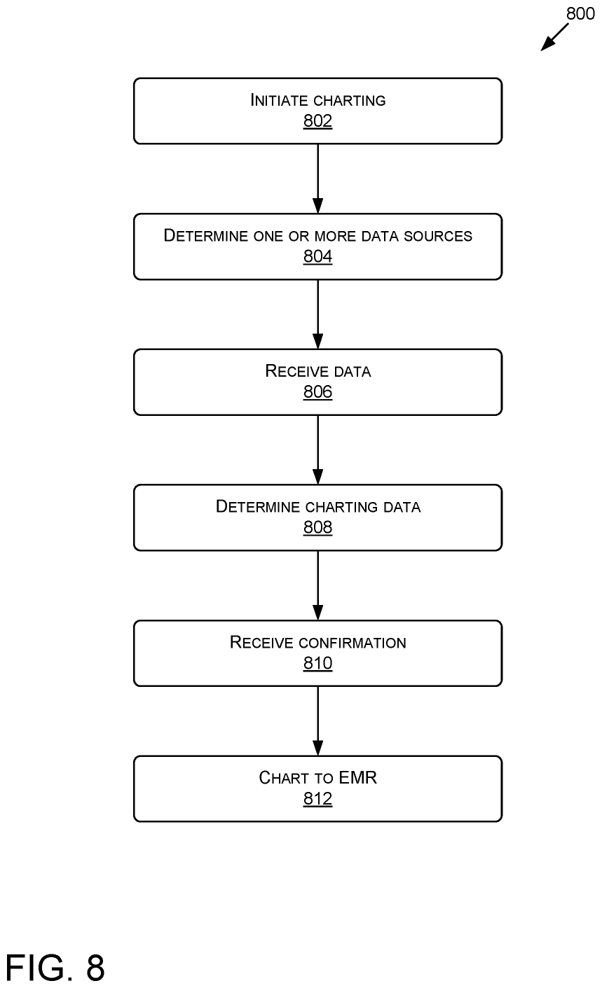

The scheduling module 326 may be configured to initiate charting and/or capture of data by the system. In an example, the scheduling module may be configured to initiate an automated charting process, such as the processes described with respect to FIGS. 6 and 8, in response to a patient being admitted into a care facility, a caregiver entering a patient room, the expiration of a regular or otherwise predetermined time interval (e.g., once per hour), the presence of a person within a patient room, the receipt of data from a device within the patient room, the detection of an event within the patient room, or other such actions/events. The scheduling module 326 may be used to receive an input, for example from a caregiver station, to initiate charting as a caregiver enters the room. The scheduling module 326 may be configured to initiate charting as directed by a caregiver, at regular and/or irregular intervals. The scheduling module 326 may access the EMR to determine when to initiate charting for a particular patient. For example, if a patient takes a medication at a first time, the scheduling module 326 may initiate charting, after a predetermined period of time has passed, to evaluate the efficacy and impact of the medication on the patient. The scheduling module 326 may be configured to cause one or more of the other modules of the computing device 302 to perform tasks as described herein.

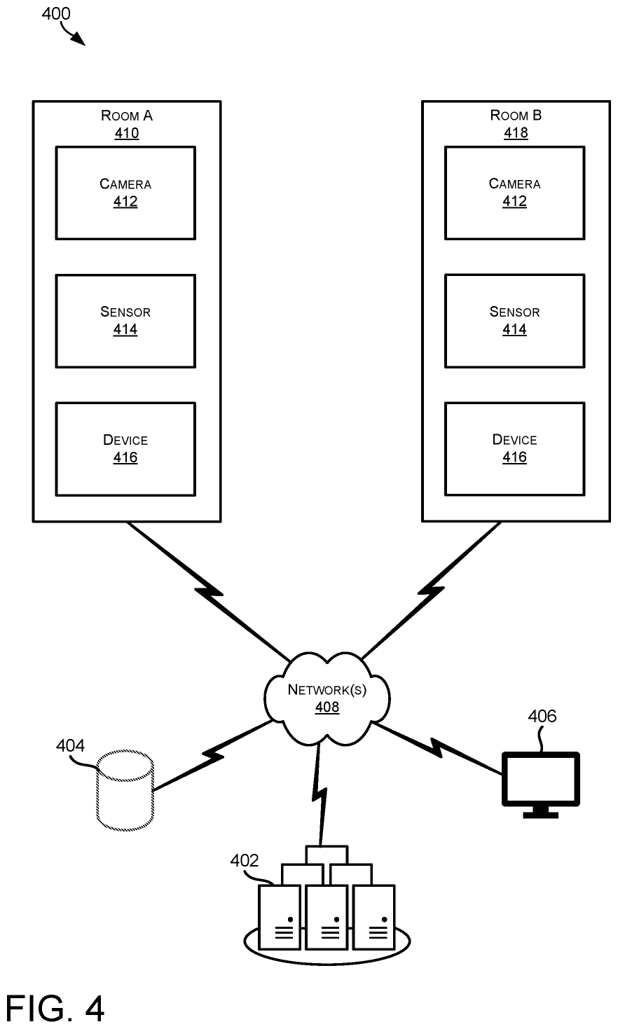

FIG. 4 illustrates an example system architecture for monitoring multiple patient areas in a patient care environment, according to at least one example. The system architecture 400 illustrates an example for monitoring, from a caregiver station 406, multiple patient rooms within a caregiver facility and using automated charting of events based on sensor data from the patient rooms.

The system architecture 400 includers one or more video cameras 412 and/or other types of vision sensing or motion sensing equipment. System architecture 400 is adapted to sense one or more conditions in a room or other environment, and/or to sense one or more actions undertaken by one or more persons in the room or other environment. The data gathered by the system architecture 400 is processed by appropriate hardware and/or software to chart to an electronic medical record and/or determine whether an alert or other type of notification should be forwarded to appropriate personnel. As described herein, system architecture 400 may be especially suited for use in a patient care environment, such as a hospital, nursing home, or other facility where patients are housed.

In some embodiments, system architecture 400 may be used to gather various information about the patient and the room in which the patient is located in order to determine events and activity to chart to the EMR for the patient and also to alert appropriate personnel of any conditions that may require attention. Events such as clinician interactions, patient movements, patient interactions with devices, entering or exiting of the room, status of equipment in the room, and other such type of information. Still other types of information may be processed and/or used—either in lieu of, or in addition to, the foregoing information—in order to provide a complete picture of patient care as part of the EMR. Still further, system architecture 400 may also provide remote broadcasting of video information to remote locations, such as a nurse's station (e.g., caregiver station), a mobile device, a website accessible to relatives of the patient, and other such locations and devices. Appropriate alarms or alerts may also be generated by system architecture 400 and forwarded to the appropriate healthcare personnel when conditions or actions are detected that require attention.

In other embodiments, system architecture 400 may be used to gather information about the patient and the patient's room which is forwarded to appropriate personnel so that the risk of spreading infection either from or to the patient is reduced. In such embodiments, system architecture 400 may detect any one or more of the following conditions: (1) whether a clinician has washed his or her hands prior to approaching or touching a patient; (2) whether one or more sterile fields within the room are maintained and/or contaminated; (3) whether personal protection equipment is being used—such as masks, gowns, gloves, and the like—by personnel who enter the room; (4) whether objects within the room are mobile or stationary, and whether an alert should be issued to the appropriate personnel for proper cleaning of the object prior to its leaving and/or entering the room; and (5) whether areas within the room have been properly and/or completely cleaned. Upon the detection of any one or more of these conditions, system architecture 400 may forward appropriate information regarding the condition to appropriate personnel. In some examples, the computing device 402 may maintain a log of locations visited by clinicians such that if a patient is later diagnosed with a communicable virus or disease that the possible route of infection may be traced to contain the spread by quarantining or limiting exposure.

In other embodiments, system architecture 400 may be used to help ensure that patient care protocols are properly followed. For example, computing device 402 may automatically detect when a clinician enters the patient's room and monitor the activities performed by the clinician to ensure that one or more desired activities are performed. computing device 402 may also be used to monitor compliance with patient care protocols. Oftentimes, for example, such patient care protocols require that a patient be turned while positioned on the bed at certain intervals so as to lessen the likelihood of bed sores and/or other medical ailments. Computing device 402 can be used to detect the absence or presence of such turning at the required intervals. Computing device 402 may also be used to determine that the head of the patient's bed remains positioned at a desired angle, or that the height of the patient's bed remains at a desired level. Maintaining the angle of the head of the bed at a desired angle may be desirable in order to lessen the likelihood of ventilator associated pneumonia, and maintaining the bed at a low height may be desirable for reducing the likelihood of patient falls when getting into or out of the bed.

In still other embodiments, system architecture 400 may be used to monitor the patient within the room and alert the appropriate caregivers of any situations that they should be made aware of. These may include detecting whether the patient is in the room, has moved to the restroom, or has exited the room altogether. Other conditions may include determining if the patient is eating, sleeping, exiting the bed, walking, having a seizure, falling, getting entrapped in side rails, sitting in a recliner, or experiencing pain. Still other information about the patient may be gathered and processed. Some or all of such information may be stored in the EMR as events that may be referred to by caregivers for reference later, for example to view when a patient last ate.

In some examples, any of the cameras described herein may comprise an RGB sensor, a digital camera, an infrared camera, a thermal camera, or other camera types that may be used to capture image data in one or more wavelength ranges. For example, such cameras may comprise a digital camera configured to capture video and/or still images (e.g., digital photographs) of/depicting the patient, medical devices, and/or other items within a field of view of the camera. In some examples, the cameras may also include a stereo camera or other camera capable of capturing and/or gathering data that may be used to determine a distance to an object in the image data.

In one embodiment, any one or more of the cameras 412 of system architecture 400 may be a motion sensing device sold under the brand name Kinect™, or variations thereof, by Microsoft Corporation® of Redmond, Wash., USA. The Kinect™ motion sensing device includes an RGB (red, green, blue) camera, a depth sensor, and a multi-array microphone. This device may be used to provide full-body 3D motion, facial recognition, and voice recognition capabilities. The depth sensor may include an infrared laser projector combined with a complementary metal oxide semiconductor (CMOS) sensor, which captures reflected signals from the laser projector and combines these signals with the RGB sensor signals. The Kinect™ motion sensing device may automatically detect the position of one or more persons and output data indicating the locations of multiple body portions, such as various joints of the person, multiple times a second. Such information may then be processed to determine any one or more of the conditions discussed herein.

In other embodiments, any one or more of the cameras 412 may be a WAVI Xtion™ motion sensing system, or variations thereof, marketed by Asustek Computer, Inc., which has a principal place of business in Taipei, Taiwan. The WAVI Xtion™ motion sensing system uses one or more depth sensors to sense the position and movement of people without requiring the people to hold any objects.

In still other embodiments, other types of cameras may be used, or a combination of one or more of the Kinect™ cameras may be used with one or more of the WAVI Xtion™ cameras along with other, typical, or traditional video cameras. Still other combinations of cameras may be used. Modifications may also be made to the camera 412, whether it includes a Kinect™ camera or a WAVI Xtion™ camera, or some other camera, in order to carry out the functions described herein, as would be known to one of ordinary skill in the art. It will further be understood that depth sensing devices may be used in system architecture 400 that are physically separate from the image sensing portion of cameras 412. The terms “camera,” as used herein, will therefore encompass devices that only detect images, as well as devices that detect both images and depths. The images detected may refer to both ambient light images or thermal images, or still other types of images.

Whatever type or types of cameras 412 that are used, such cameras 412 may include additional sensors beyond the image sensors and/or depth sensors, such as microphones, or other sensors. In some embodiments, it may be desirable to utilize more than one camera 412 within a room 410 or 418, or more than one camera 412 for a given patient. The use of multiple cameras for a given room or patient may decrease the likelihood of the camera's view being obstructed, and may increase the different types of information that may be gathered by the cameras 412. When multiple cameras 412 are used within a given room or for a given patient, the cameras 412 may all be of the same type, or they may consist of different types of cameras (e.g., some cameras may include both image sensors and depth detectors while others may only have image sensors).

Sensor 414 may include sensors beyond optical sensors, such as sensors of medical devices, depth sensors, proximity sensors, and other sensor for detecting specific types of activities may be implemented in the rooms 410 and 418 in addition to the cameras 412. Furthermore, devices 416, such as medical devices, equipment, beds, and other such components within a caregiver facility may be positioned within the rooms 410 and 418 and capable of sending patient data regarding events and patient status to the computing device 402.

The cameras 412, sensors 414, and devices 416 that are positioned within a given room, or other location, are in electrical communication with the computer device 402 via a communications medium, such as, but not limited to, network 408, which may be a local area network (network), a wide area network (WAN), or any other type of network, including a network that is coupled to the Internet. Network 408 may be an Ethernet-based network, or other type of network. The cameras 412 are positioned within a patient care facility, such as a hospital, nursing home, or the like, and record images of various activity. Such images are converted to electrical signals which are forwarded to computing device 402 for processing in various manners, as described herein.

Computing device 402 may be a conventional server that communicates with cameras 412, sensors 414, and devices 416 over network 408, or it may be one or more personal computers (PCs), or it may be a dedicated electronic structure configured to carry out the logic and algorithms described herein, or any combination of these or other known devices capable of carrying out the logic and algorithms described herein. Such dedicated electronic structures may include any combination of one or more processors, systems on chip (SoC), field programmable gate arrays (FPGA), microcontrollers, discrete logic circuitry, software and/or firmware. Regardless of whether computing device 402 is a single physical device, or is multiple physical devices working together (which may be located in different physical locations), computing device 402 represents the hardware, software and/or firmware necessary to carry out the algorithms described herein.

The computing device 402 may also communicated with database 404 and caregiver station 406, similar to database 306 and caregiver station 106, respectively.

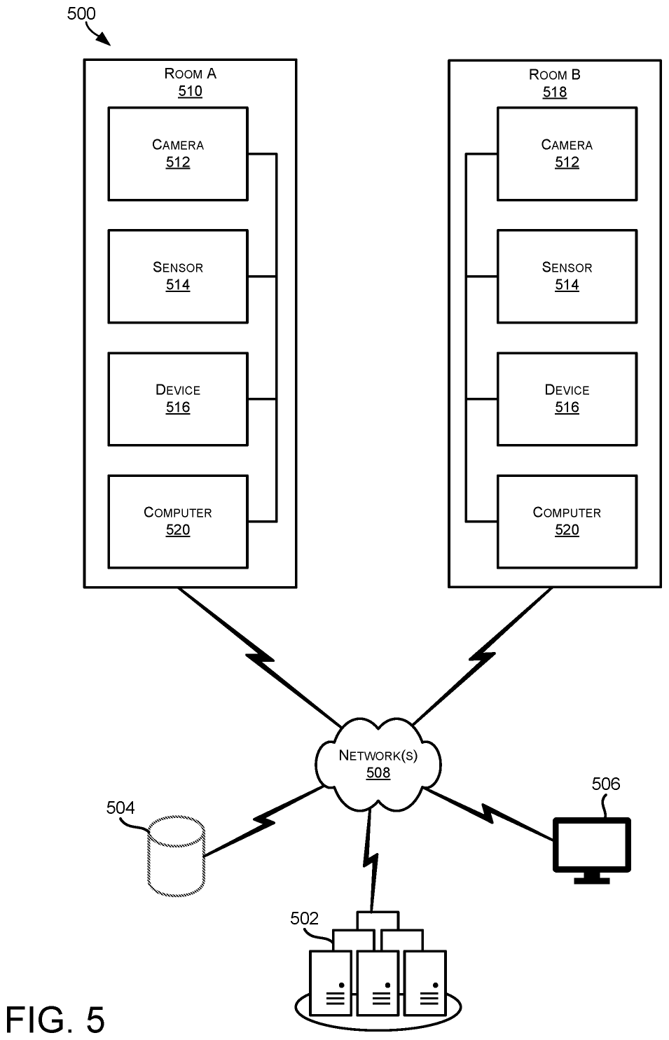

FIG. 5 illustrates a system architecture 500 for monitoring and centralizing patient care data, according to at least one example. In some examples, the system architecture 400 of FIG. 4 may be arranged with a semi-distributed computing network, with computing devices locally in or near the rooms. The system architecture 500 includes components similar or identical to those of FIG. 4, such as the computing device 502, database 504, caregiver station 506, room 510, camera 512, sensor 514, device 516, and room 518 which may correspond to the computing device 402, database 404, caregiver station 406, room 410, camera 412, sensor 414, device 416, and room 418 of FIG. 4.

In the system architecture of FIG. 5, a computing device 520 is positioned in each room that is dedicated to processing the images and/or depth sensor readings generated by the cameras 512 and other sensors positioned within that room. After processing all or a portion of the data received from the cameras 512, the in-room computing devices 520 may transmit messages regarding such processing onto the network 508. Such messages may be sent to the computing device 502 for further processing or, alternatively, such messages may be forwarded directly to one or more other computer devices that are in communication with network 508, such as, but not limited to, an electronic medical records (EMR) computer device, a work flow management computer device, a caregiver alerts computer device, an admissions, discharge, and transfer (ADT) computer device, or any other computer device in communication with network 508.

In another alternative embodiment (not shown), each camera 512, sensor 514, and/or device 516 may include its own computing device or its own portion of a computing device, either separately attached thereto, or integrated into the camera 512 itself. In such an embodiment, each computing device is dedicated to processing, or pre-processing, the electronic images, depth sensor readings, and/or voice signals gathered by the associated camera 512. The results of such processing, or pre-processing, may then be forwarded directly to network 508, or to one or more intermediate computers (not shown) before being sent to network 508. Computer devices provide the software intelligence for processing the images, depth sensor data, and/or voice data recorded by cameras, and the precise physical location of this intelligence can vary in a wide variety of different manners, from embodiments in which all the intelligence is centrally located to other embodiments wherein multiple computing structures are included and the intelligence is physically distributed throughout the caregiving facility.

The database 504 may contain information that is useful for one or more of the algorithms carried out by system architecture 500. This information may include photographic and/or other physical characteristic information of all of the current clinicians and/or staff of the patient care facility so that computing device 502 can compare this information to the signals detected by cameras 512 to identify if a person is a hospital employee and/or who the employee is. This information may also include photographic and/or other physical data of the current patients within the patient care facility so that patients can be recognized by computing device 502. The information within database 504 may also include data that is specific to individual rooms within the facility, such as the layout of the room, the location of restrooms, where and what objects are positioned within the room, the dimensions of the room, the location of room doors, the heights of floors, suitable or designated locations within the rooms for placing signs, and other useful information. The database may also include identifying information for identifying objects and assets, such as equipment used within the patient care facility. Such identifying information may include information about the shape, size, and/or colors of objects that computing device is designed to detect. Still other information may be included within database 504.

The system architecture 500 is configured to detect people who appear in the images detected by cameras 512. The detection of such people can be carried out in known manners, as would be known to one of ordinary skill in the art. In at least one embodiment, computing device 502 detects such people and generates a rudimentary skeleton that corresponds to the current location of each individual detected by cameras 512.

In general, cameras 512 may be positioned to record image information useful for any one or more of the following purposes: ensuring proper patient care protocols are followed; identifying the type of behavior of a patient or the patient's condition; reducing the risk of infection and/or assisting in the containment of possible infectious agents; and/or taking measures to either reduce the likelihood of a patient falling, or to respond to a patient quickly after a fall has occurred.

The system architecture 500 may be used to help ensure that patient care protocols used by a caregiver facility are followed. Depending upon the condition of the patient, different care protocols may be implemented in order to provide care that is optimally tailored to that patient. System architecture 500 may be used in a variety of different manners for helping to ensure these protocols are properly followed. In some embodiments, computing device 502 may recognize behaviors of a clinician and forward information about that behavior to the appropriate hospital computer or server.

FIG. 6 illustrates a process 600 for charting patient information to an electronic medical record using the systems described herein, according to at least one example. The process is illustrated as a collection of blocks in logical flow diagrams, which represent a sequence of operations, some, or all of which may be implemented in hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed.

At 602, the process 600 includes a computing device, such as the computing device 102 initiating charting for a patient, for example in response to a patient being admitted into a hospital or other caregiving environment. In some examples, the computing device 102 may initiate charting at a regular time interval, in response to sensor data received from equipment, or in response to detecting a presence of a caregiver in the room. For example, when the caregiver enters, the system may identify the caregiver using cameras within the room.

After initiating charting, the process 600 may include the computing device 102 determining if additional devices are needed at 604, for example including devices to gather sensor data, medical devices, equipment, or other such components. In the event that no additional devices are needed, the process proceeds to step 610 and relies on data from the cameras within the room.

In the event that an additional device is needed, the device is identified by the computing device 102 at 606, when possible. When not possible, the process 600 may include determining, by the computing device 102 if a clinician is in the room who can find the device at 624 and then produces an alert to the caregiver at 626, to a device of the caregiver, a device in the room, or other such alert system. The caregiver may be identified by an object or person recognition algorithm running on video data from the cameras. The identity may be accessed as described with respect to FIG. 1 above. If the device is found, then the process 600 includes connecting to the device to receive sensor data at 608. Again, if the device is unable to connect, then an alert is issued to the caregiver to aid with connectivity issues.

After alerting to the connection issue with the device, the process 600 may proceed with the caregiver fixing the obstruction at 628 or the computing device 102 determining a different camera to use that is not obstructed. When resolved at 632, the process 600 can return to 602 with the additional device now connected. In the event that the obstruction or connection to the device is not fixable at 628, the computing device 102 may initiate charting at 630 without using the device, and will proceed as if no additional device is needed at 604. In some examples, the charting at 630 may include voice charting, for example to chart information to the EMR based on information spoken or dictated by the caregiver. In some examples, the voice charting may be initiated when attempts to resolve obstructions have failed and the caregiver is present within the room. In examples including the voice charting, the dictated information may be confirmed at the caregiver station or other location to provide redundancy and risk reduction.

Returning to step 610, the process 600 includes the computing device 102 determining whether the patient is visible in the image data. The computing device 102 may determine if the patient is visible by running a person detection algorithm on the image data, and by uniquely identifying the patient based on a code, visible identifier, or other technique, such as described with respect to FIG. 1. If the patient is not visible then the system may delay for a time period at 634, but after a threshold period of time will alert the caregiver at 636 that the patient is not visible to the charting system so the caregiver can address the visibility.

With the patient visible at step 610, the computing device 102 records reference data and a timestamp associated with the data at step 612. The reference data may include video data, sensor data, data from an additional device (if connected), or other such information. In some examples, a single reference frame may be captured from the video data for audit purposes. After recording reference data, the computing system 102, using one or more machine learning algorithms, determines charting data at step 614. The charting data includes event identification, identification of individuals, identification of devices, sensor data, and other data that may be recorded to the EMR. The charting data may include annotation generated by the computing device in response to the gathered data.

At step 616, the process 600 include the computing device 102 determining if review is needed for the charting data. Some data may require verification, for example if related to an especially sensitive topic or particular set of procedures or medications that guidelines require verification for health and safety purposes. The review may also be a result of confidence in the charting data, as described herein. The confidence score may be below a threshold resulting in a requirement for review at 616 that places the charting data into a review queue at 618. If no review is required, the computing system 102 can write the charting data to the EMR at 622. After the review, by a caregiver or designated reviewer, the charting data may be confirmed at 620 for charting at 622 or rejected, which may result in re-starting the charting process back to step 602.

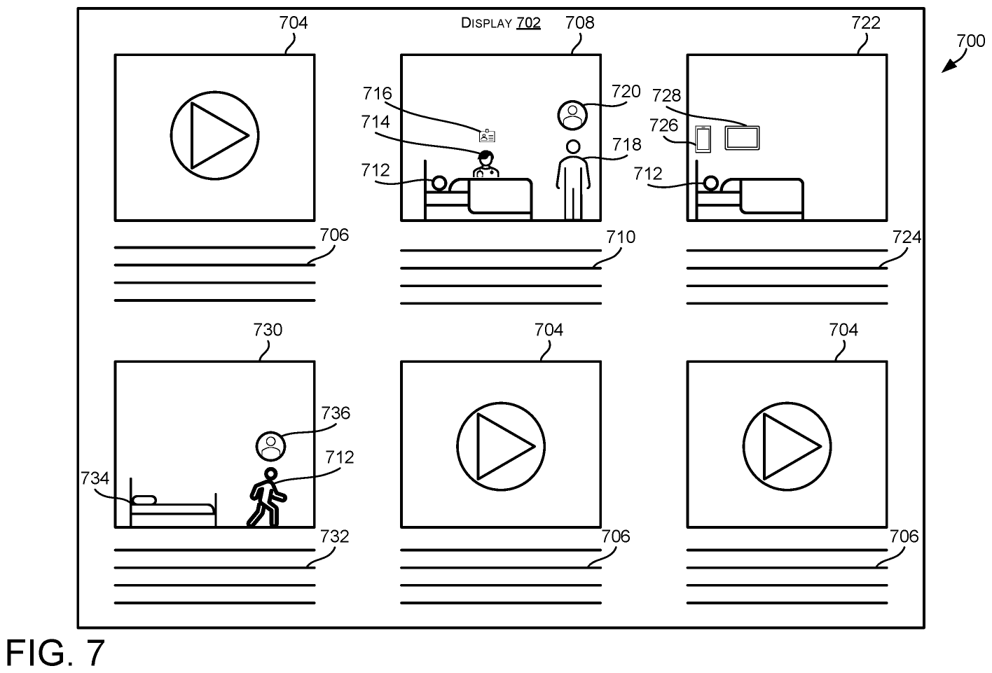

FIG. 7 illustrates an example user interface 700 for a patient care monitoring hub, according to at least one example. The example user interface 700 includes a display 702 that may represent a view at a caregiver station in a caregiver facility. The display 702 includes multiple sections for different patients that are monitored by the caregiver station. The video 704 for several of the rooms may be paused, turned off (due to vacancy), or otherwise unavailable. In some examples, the video 704 may not show in real-time but may show when there is an event to review. In some examples the video 704 may show outside of times when a patient requests privacy or the room is unoccupied. Corresponding data from an EMR 706 is displayed beneath the video 704.

In a second pane of the display 702, video 708 illustrating a caregiver 714 and individual 718 in the room of a patient 712 is shown. An identification 716 for the caregiver is recognized by the system such that the caregiver is recognized. The identity of the individual 718 may be known or unknown, as described with respect to FIGS. 1-2, in either case a unique identifier 720 is assigned to the individual 718. EMR data 710 is displayed beneath the video 708 that may represent an event charted or awaiting review corresponding to the event of the video 708.

In another pane of the display 702, a patient 712 is shown with devices 726 and 728 visible in the video 722. The event recorded in the EMR data 724 may correspond to data visible on the displays of the devices 726 and 728 that may be recorded in the EMR as an event.

In another pane of the display, the patient 712 is shown getting out of bed 734 and walking around the room in video 730. The identity of the patient is known and represented by the identifier 736. The event may be logged in the EMR data 732 associated with the video 730.

In some examples, the display 702 may have other arrangements and configurations, the display 702 is meant to be illustrative only and represent types of information and data that may be presented at the caregiver station using the systems herein. The caregiver may interact with an input device to confirm event data, as described above through the display 702.

FIG. 8 illustrates a process 800 for automatic charting of patient information to an electronic medical record, according to at least one example. The process 800 is illustrated as a collection of blocks in logical flow diagrams, which represent a sequence of operations, some, or all of which may be implemented in hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed.

At 802, the process 800 includes a computing device initiating charting. At 802, the computing device may initiate the charting in response to one or more detected events within the room, such as a person entering, detection of movement, detection of a caregiver or clinician in the room, detection of information from a device in the room, or other such detected events. The detection may be through a camera capturing image data or may be through one or more other sensors, such as an audio sensor picking up sounds or instructions to initiate charting. The computing device may initiate charting in response to a manual input, such as a wake word, clicking a button on a user input device, or other manual input to the system. The computing device may also initiate charting at a set interval, such as at regular one-hour intervals. The computing device may initiate the charting by using the scheduling module 326 as described with respect to FIG. 3.

At 804, the process 800 includes the computing device determining one or more data sources. The data sources include one or more cameras within the patient room as well as other potential sources of data for charting information to the EMR of the patient. The one or more data source may include devices to gather sensor data, medical devices, equipment, or other such components. At 804, the computing device may determine one or more such data sources (e.g., a device or system) based on a determination that the system or device is in use and/or associated with an event taking place in the patient room.

At 806, the process 800 includes the computing device receiving data from the one or more data sources. The data may be received directly from the one or more data sources, for example through a network, wireless connection, or other such data connection that may pass through one or more intermediate devices. In some examples, the data may be received by determining data based on image data, such as by determining data displayed on a patient monitor and using one or more language recognition techniques to determine the data displayed on the monitor. In this manner, the system may receive data from devices that are not connected to the computing device, or may be incompatible with the system and/or computing device, thereby enabling the use of additional devices and systems that otherwise would not operate within the caregiver facility due to lack of compatibility.

At 808, the process 800 includes the computing device determining charting data. The charting data may be generated using one or more machine learning algorithms as described herein. The charting data includes event identification, identification of individuals, identification of devices, sensor data, and/or other data that may be recorded to the EMR. The charting data may further include one or more annotations generated by the computing device in response to the gathered data.

At 810, the process 800 includes the computing device receiving confirmation of the charting data. In some examples, the confirmation may be a manual confirmation of the charting data by a caregiver. Such confirmed data may be used for continuous learning and refinement of the machine learning models used to generate the charting data. Some charting data, for example based on the content of the data being of low importance, low risk, or otherwise classified as not requiring confirmation, may not require confirmation but may proceed to charting at 812. In some examples, the charting data may be associated with a confidence score reflective of confidence that the charting data reflects the event within the patient room. In some examples, the confirmation may only be required in instances where the confidence score falls below a threshold value.

At 812, the process 800 includes the computing device charting the data to the EMR. The computing device can write the charting data to the EMR for the patient. After the review, by a caregiver or designated reviewer, the charting data may be confirmed at 810 for charting at 812 or rejected, which may result in re-starting the charting process.

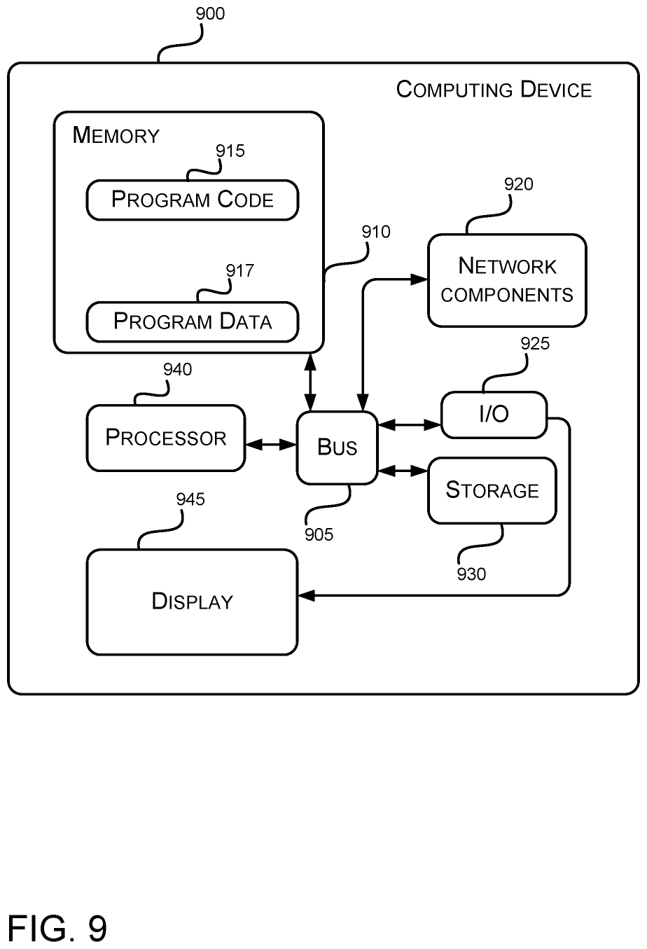

FIG. 9 illustrates a block diagram of an example of a computing device 900. Computing device 900 can be any of the described computers herein including, for example, computing device 102 of FIG. 1, computing device 302 of FIG. 3, computing device 402 of FIG. 4, computing device 502, computer A 526, and computer B 528 of FIG. 5. The computing device 900 can be or include, for example, an integrated computer, a laptop computer, desktop computer, tablet, server, or other electronic device.

The computing device 900 can include a processor 940 interfaced with other hardware via a bus 905. A memory 910, which can include any suitable tangible (and non-transitory) computer readable medium, such as RAM, ROM, EEPROM, or the like, can embody program components (e.g., program code 915) that configure operation of the computing device 900. Memory 910 can store the program code 915, program data 917, or both. In some examples, the computing device 900 can include input/output (“I/O”) interface components 925 (e.g., for interfacing with a display 945, keyboard, mouse, and the like) and additional storage 930.

The computing device 900 executes program code 915 that configures the processor 940 to perform one or more of the operations described herein. Examples of the program code 915 include, in various embodiments logic flowchart described with respect to FIG. 6 or FIG. 8 above. The program code 915 may be resident in the memory 910 or any suitable computer-readable medium and may be executed by the processor 940 or any other suitable processor.

The computing device 900 may generate or receive program data 917 by virtue of executing the program code 915. For example, sensor data, trip counter, authenticated messages, trip flags, and other data described herein are all examples of program data 917 that may be used by the computing device 900 during execution of the program code 915.

The computing device 900 can include network components 920. Network components 920 can represent one or more of any components that facilitate a network connection. In some examples, the network components 920 can facilitate a wireless connection and include wireless interfaces such as IEEE 802.11, BLUETOOTH™, or radio interfaces for accessing cellular telephone networks (e.g., a transceiver/antenna for accessing CDMA, GSM, UMTS, or other mobile communications network). In other examples, the network components 920 can be wired and can include interfaces such as Ethernet, USB, or IEEE 1394.

Although FIG. 9 depicts a computing device 900 with a processor 940, the system can include any number of computing devices 900 and any number of processor 940. For example, multiple computing devices 900 or multiple processor 940 can be distributed over a wired or wireless network (e.g., a Wide Area Network, Local Area Network, or the Internet). The multiple computing devices 900 or multiple processor 940 can perform any of the steps of the present disclosure individually or in coordination with one another.

Other embodiments of description will be apparent to those skilled in the art from consideration of the specification and practice of the examples described herein. It is intended that the specification and examples be considered as example only, with a true scope and spirit of the present disclosure being indicated by the following claims.

What is claimed is:

1. A system, comprising:
a processor; a non-transitory, computer-readable media having instructions stored thereon that, when executed by the processor, cause the processor to perform acts comprising:
receiving video data from a camera operably connected to the processor, the video data being associated with a patient disposed in the room;
determining, using a trained machine learning model, an occurrence of an event within the room and associated with the patient;
generating, using the trained machine learning model, an annotation indicative of the event, the annotation comprising a description of the event and associated patient data; and
updating, based on the event and the annotation, an electronic medical record of the patient.

2. The system of claim 1, wherein the trained machine learning model is trained using data including training video data of patient care facilities with associated annotations describing events represented within the training video data.

3. The system of claim 1, wherein determining the occurrence of the event comprises determining an identity of a person in the room, and wherein the annotation indicates the identity of the person.

4. The system of claim 3, the acts further comprising:
receiving patient data from one or more medical devices associated with the patient, the patient data comprising patient vital data comprising at least one of temperature, blood pressure, heart rate, blood oxygenation, or movement information; and
updating the electronic medical record based on the patient data, and wherein the annotation is generated based on the patient data.

5. The system of claim 4, wherein receiving the patient data comprises:
determining, based on the video data, that a display associated with the one or more medical devices is presenting a representation of the patient data; and
determining the patient data from the video data and the representation of the patient data.

6. The system of claim 1, wherein determining the event comprises determining a care procedure, and wherein the acts further comprise:
determining, based on the electronic medical record, a prescribed procedure for the patient;
determining a compliance score based on the event and the prescribed procedure; and
updating the electronic medical record based on the compliance score.

7. The system of claim 1, wherein the camera comprises a first camera, the video data comprises first video data, the room comprises a first room, the event comprises a first event, the annotation comprises a first annotation, the patient comprises a first patient, and the system further comprises a second camera positioned in a second room of the patient care facility, the acts further comprising:
receiving second video data from the second camera, the second video data being associated with a second patient disposed in the second room;
determining, using the machine learning model, a second event occurring within the second room;
generating, using the trained machine learning model, a second annotation associated with the second event; and
updating, based on the second event and the second annotation, the electronic medical record of the second patient.

8. The system of claim 7, further comprising a caregiver station comprising a display and an input device, and wherein the acts further comprise displaying, at the display of the caregiver station, first video data representing the first event, the first annotation, second video data representing the second event, and the second annotation.

9. The system of claim 8, wherein the acts further comprise receiving an input via the input device, and wherein at least one of the electronic medical record of the first patient or the electronic medical record of the second patient, is updated based on the input.

10. The system of claim 9, wherein the acts further comprise:
determining, using the trained machine learning model, a confidence score associated with the first event;
determining that the confidence score is below a confidence score threshold;
generating a request for a user input based on the confidence score being below the confidence score threshold; and
presenting the request via the display of the caregiver station.

11. A method, comprising:
receiving, at a computing device associated with a care facility, video data from a camera positioned within a patient room of the care facility;
determining, using a trained machine learning model, an occurrence of an event within the patient room and associated with a patient;
generating, using the trained machine learning model, an annotation indicative of the event, the annotation comprising a description of the event and patient data; and
updating, based on the event and the annotation, an electronic medical record of the patient.

12. The method of claim 11, further comprising displaying at a caregiver station of the care facility, a representation of the video data associated with the event, the electronic medical record, and the annotation.

13. The method of claim 11, further comprising
determining a confidence score associated with the event;
determining that the confidence score is below a confidence score threshold;
generating a request for a user input based on the confidence score being below the confidence score threshold; and
presenting the request via a display of the care facility.

14. The method of claim 11, wherein determining the occurrence of the event comprises determining an identity of a person in the patient room, and wherein the annotation indicates the identity of the person.

15. The method of claim 14, wherein determining the identity of the person comprises:
accessing the video data;
determining a unique identifier associated with the person visible in the video data; and
determining the identity of the person based on the unique identifier.

16. The method of claim 15, further comprising:
determining tracking data for the person within the patient room based on the video data; and
determining an interaction between the person and the patient or equipment associated with the patient based on the tracking data, and wherein the patient data of the annotation comprises data representing the interaction.

17. One or more non-transitory computer-readable media having instructions stored thereon that, when executed by one or more processors, cause the one or more processors to perform operations comprising:
receiving, at a computing device associated with a care facility, video data from a camera positioned within a patient room of the care facility;
determining, using a trained machine learning model, an occurrence of an event within the patient room and associated with a patient;
generating, using the trained machine learning model, an annotation indicative of the event, the annotation comprising a description of the event and associated patient data; and
updating, based on the event and the annotation, an electronic medical record of the patient.

18. The one or more non-transitory computer-readable media of claim 17, wherein determining the event comprises determining a care procedure, and wherein the operations further comprise:
determining, based on the electronic medical record, a prescribed procedure for the patient;
determining, based on the event, a compliance score based on the event and the prescribed procedure; and
updating the electronic medical record based on the compliance score.

19. The one or more non-transitory computer-readable media of claim 17, the operations further comprising:
receiving patient data from one or more medical devices, the patient data comprising patient vital data; and
updating the electronic medical record based on the patient data, and wherein generating the annotation is further based on the patient data.

20. The one or more non-transitory computer-readable media of claim 19, wherein receiving the patient data comprises:
determining, based on the video data, that a display associated with the one or more medical devices is presenting a representation of the patient data; and
determining the patient data from the video data and the representation of the patient data.
